(19) United States
(12) Patent Application Publication
(10) Pub. No.: US 2026/0124465 A1
(43) Pub. Date: May 7, 2026

(54) WEARABLE RADIATION EMITTING THERAPY DEVICE FOR AN AMPUTEE PATIENT

(71) Applicant: Eduardo Marti, Weston, FL (US)

(72) Inventor: Eduardo Marti, Weston, FL (US)

(21) Appl. No.: 18/935,470

(22) Filed: Nov. 2, 2024

(51) Int. Cl. A61N 5/06

(52) U.S. Cl. CPC A61N 5/0624; A61N 2005/0645; A61N 2005/0652; A61N 2005/0659; A61N 2005/0663

(57) ABSTRACT

A wearable radiation emitting therapy device for an amputee patient which is structured to emit at an amputation site a spectrum of near infrared light and infrared light suitable for promoting recovery, reducing general pain, and preventing phantom limb pain syndrome in post op scenarios for amputee patients. The wearable radiation emitting therapy device may be formed as a wrap body suitable to enclose the stump and surrounding areas of an amputated limb, with the having a pair of elastic securing straps in a proximal position relative to the stump and a wraparound adjustment strap in a distal position relative thereto. A control module releasably attachable to the wrap body, with the control module operative to wirelessly control two or more types of LED lights positioned inside the wrap body so as to cause the lights to direct various spectrums of light into and around the amputation site.

### BACKGROUND OF THE INVENTION

### Field of the Invention

This invention relates generally to wearable therapeutic devices and, more particularly, to a device which generates therapeutic radiation at a plurality of wavelengths and is worn to continually direct said radiation into a targeted part of a wearer's body.

### Description of the Prior Art

Amputation, commonly understood as the removal of a limb or other body part by medical illness, trauma, or surgery, is a practice that is known to date back in humans at least 31,000 years and remains well established in many cultures. While amputation is a practice that has in some cases been performed in a ritualistic or punishment context, in modern times it's most commonly seen in a medical context. In this regard, amputation is often employed as a result of injury or disease to try to avoid a more serious outcome.

After amputation of a limb is executed, whether because of a medical illness, trauma or surgery, it is well established in modern medicine that the amputation site will require post operative treatment. This treatment can take on various forms, and can include pain management, infection prevention and rehabilitation of the injury site. When done effectively, post operative treatment has been shown to improve the success rate of the surgical site to heal and recover correctly without secondary issues

While there are various post operative treatment methods for amputees, it has been found that visible and infrared light emitting diode (“LED”) therapy can provide a range of benefits with relative ease of application. Indeed, the application of various specific spectrums of visible and infrared light has been shown to promote blood flow and oxygenation to the site and therefore increase the rate at which the site heals while reducing scarring. Light spectrums such as near infrared blue light have also been shown to reduce bacterial infection by killing unwanted bacteria such as Staphylococcus virus, which is a major risk factor in post-op scenarios.

As a result of improved healing, the amount of pain experienced by the patient can be reduced. The effects of the various infrared light spectrums also reduce the likelihood of conditions such as phantom limb pain syndrome, which is often triggered by blood flow issues to the amputation site, excessive swelling or infection.

In addition, the noninvasive nature of radiation therapy also can make it well suited for a patient to be able to conduct it themselves or with assistance at various stages of the rehabilitation process.

Accordingly, there remains a need for a radiation emitting therapy device specifically designed to be worn by amputee patients in a way that directs radiation into an amputation site. It would be desirable for such a radiation emitting therapy device to include LEDs operable to emit the most effective spectrums of light for aiding the recovery process, such as near spectrum red and blue light, as well as infrared light. It would additionally be desirable for such a therapy device to be easily fitted over and comfortable to wear at the site of an amputation.

### SUMMARY OF THE INVENTION

The present disclosure provides for a wearable radiation emitting therapy device for an amputee patient, comprising: a device body having an open top defining a proximal end, a closed bottom defining a distal end, and a plurality of side walls, wherein the device body includes an interior surface which extends across the enclosed bottom and at least a portion of the plurality of side walls; wherein the device body is adapted to be positioned over an amputated member having an amputation site such that the interior surface is adjacent to at least the amputation site; a plurality of electrical radiation emitting aspects positioned on the interior surface and configured to selectively generate radiation when activated, wherein the plurality of radiation emitting aspects are arranged such that when activated, the plurality of radiation emitting aspects direct radiation into the amputated member at the amputation site and into at least one area on the amputation member adjacent to the amputation site; at least one adjustment strap integral with the device body, wherein the at least one adjustment strap is configured to tighten the device body on the amputated member.

Embodiments of the wearable radiation emitting therapy device for an amputee patient may additionally comprise at least one securing strap integral with the device body, wherein the at least one securing strap is disposed between a first side wall among the plurality of side walls and a second side wall among the plurality of side walls at a location distinct from the location of the at least one adjustment strap.

It is an object of the present disclosure to provide a radiation emitting therapy device specifically designed to be worn by amputee patients in a way that directs radiation into an amputation site.

It is an additional object of the present disclosure to provide a radiation emitting therapy device which includes LEDs operable to emit the most effective spectrums of light for aiding the recovery process, such as near spectrum red and blue light, as well as infrared light.

It is an additional object of the present disclosure to provide a therapy device to be easily fitted over and comfortable to wear at the site of an amputation.

### BRIEF DESCRIPTION OF THE DRAWINGS

FIG. 1 is a front elevational view of a wearable radiation emitting therapy device for an amputee patient built in accordance with the present disclosure, shown in place on a wearer.

FIG. 2 is a front perspective view of a wearable radiation emitting therapy device for an amputee patient built in accordance with the present disclosure.

FIG. 3 is a top plan view of the internal LED layout for a wearable radiation emitting therapy device for an amputee patient built in accordance with the present disclosure.

FIG. 4 is a graph showing an exemplary single wavelength output for LEDs in a wearable radiation emitting therapy device for an amputee patient built in accordance with the present disclosure.

FIG. 5 is a graph showing an exemplary multiple wavelength output for LEDs in a wearable radiation emitting therapy device for an amputee patient built in accordance with the present disclosure.

### DETAILED DESCRIPTION OF THE INVENTION

Described herein is a wearable radiation emitting therapy device for an amputee patient which is structured to emit at an amputation site a spectrum of near infrared light and infrared light suitable for promoting recovery, reducing general pain, and preventing phantom limb pain syndrome in post op scenarios for amputee patients. The wearable radiation emitting therapy device may use two types of LEDs to produce the various spectrums of light. This may include a 470 nm wavelength blue LED on one hand, and a three-in-one LED that emits a 660 nm wavelength red light, 830 nm wavelength infrared light, and 950 nm wavelength infrared light on the other.

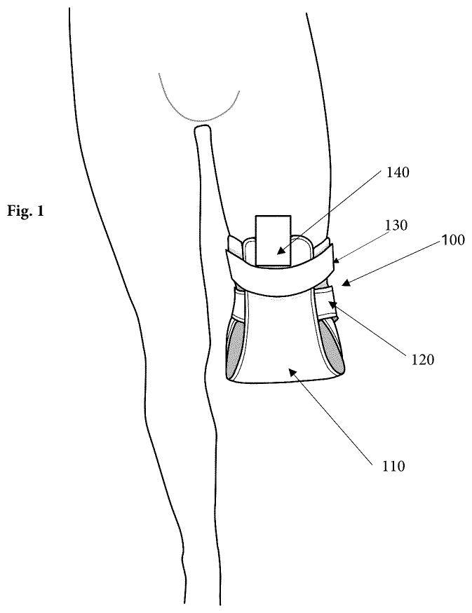

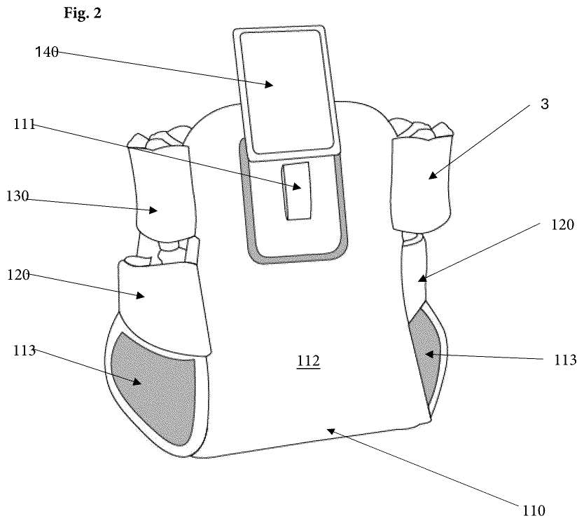

Referring now to the drawings and, in particular, FIGS. 1 and 2, a wearable radiation emitting therapy device 100 for an amputee patient is shown as a wrap body 110 having a pair of securing straps 120 and an adjustment strap 130. A control module 140 operative to control radiation emitting elements inside the wrap body 110 may be releasably attachable to an exterior surface of the wrap body 110. Embodiments of the wrap body 110 may include a molded clip 111 structured to receive a catch (not shown) on the control module 140 so as to enable the control module 140 to attach to the wrap body 110.

The wrap body 110 may be defined by a wearable wrap which has an open top, mirror image wide panels on its front side and back side 112, and mirror image narrow panels on its right side and left side 113, with the intersection of the mirror image wide panels 112 and mirror image narrow panels 113 forming an enclosed bottom. It is appreciated that the open top allows for the insertion of an amputated member into the wrap such that a terminal end of the amputated member, which has become the base of the limb, may be positioned against the bottom with the areas adjacent to the stump being surrounded by the sides of the wrap body 110.

Integral with the wrap body 110 is a pair of securing straps 120. The securing straps 120, which may be constructed of a stretchable fabric, may each be positioned to extend between the front side panel 112 and back side panel 112, right above the right side panel 113 or left side panel 113 (with one strap 120 on the left side and the other on the right side). Through their construction and positioning, the securing straps 120 helps attach the wrap body 110 to the limb (such as a leg) of a user, keeping it fitted securely to the limb and working to limit it from moving around. In addition, the securing straps 120 allow the wrap body 110 above the right side panel 113 or left side panel 113 to expand its width as needed to allow it to tightly fit on stumps and limbs of varying sizes.

Integral with the wrap body 110 above the securing straps 120 is the adjustment strap 130. The adjustment strap 130 may be defined as a wrap around strap that includes corresponding portions of a hook and loop fastener so as to allow the adjustment strap 130 to be wrapped tightly around the wrap body 110 be selectively secured to itself in a manner which tightens the wrap body 110 on the amputated member. In this regard, the adjustment strap 130 helps the wrap body 110 be securely attached to stumps and limbs of varying sizes.

The wrap body 110 may be constructed of nylon neoprene with binding on all edges so it is comfortable to wear and not cause any abrasion on the skin. The securing straps 120 may also be constructed of nylon neoprene or other elastic fabric. The adjustment strap 130 may be constructed of nylon neoprene or a strong material like polypropylene webbing so as to provide a secure hold when secured to itself.

The four (4) panel design of the wrap body 110 (with shorter side panels), the use of discrete securing straps 120 and a discrete adjustment strap 130, and the construction materials each allow for sufficient airflow to be able to reach inside the wrap body 110 so as to prevent excess perspiration and heat build-up. In addition, the binding on all raw edges of material operates to prevent discomfort due to loose fibers or edges rubbing against the patient,

The control module 140 may be constructed of rigid housing made of injected plastic and may include therein a controller, a power source such as a battery, and a wireless communication module such as a radio frequency transmitter, with each of these items electrically interconnected such that the controller can use power from the power source to generate electrical signals and send electrical signals it has generated wirelessly to a receiver using the wireless communication module. The housing may be formed with rounded edges to prevent any scratching or injury.

Referring now to FIG. 3, the wearable radiation emitting therapy device may deploy as its radiation emitting aspects LED lights arranged as a set of LED lights 150 which may be positioned on the interior surface of the wrap body, fitted facing inward on the front side panel 112f, back side panel 112b, right side panel 113r, and left side panel 113l. The set of LED lights 150 may be electrically connected to an electrical switching device, a power source, such as a battery, and a wireless receiver, such as a radio frequency receiver, so as to allow the switching device to selectively supply and remove electrical power to all of the lights in the set of LED lights from the power source in response to electrical signals received by the wireless receiver.

The set of LED lights 150 may be arranged in a cross formation, separated into six (6) discrete regions. In this regard, embodiments of the wearable radiation emitting therapy device may include two (2) regions of LEDs extending up the front side panel 112f from where the front side panel 112f extends away from the back side panel 112b, two (2) regions of LEDs extending up the back side panel 112b from where the back side panel 112b extends away from the front side panel 112f, one (1) region of LEDs on the right side panel 113r, and one (1) region of LEDs on the left side panel 113l.

It is contemplated that in such an arrangement, the two (2) regions of LEDs closest to the middle of the formation, namely the region on the front side panel 112f closest to the back side panel 112b and the region on the back side panel 112b closest to the front side panel 113, are positioned to be focused on the base of the limb. That said, because there are four (4) other regions which may respectively be directed towards the front and back of the leg, and on either side of the leg, when the wearable radiation emitting therapy device is in place on a wearer, the area being treated is not only the amputation site but the area around the amputation site (such as the area immediately adjacent to the amputation site moving away from the amputation site) also to ensure holistic treatment of the member and further reduce any issues surrounding the amputation site.

It is appreciated that the LED light therapy, particularly using a variety of wavelengths of light, assists in various ways, such as promoting blood circulation, which in turn improves oxygenation. It also assists in detoxification and an influx of antibodies to the region. Other benefits include improved scar healing and general skin healing through increased collagen production. Furthermore, as certain wavelengths have antibacterial properties, such that the blue light emits, these assists in killing bacteria including Staphylococcus and other skin bacteria.

For example, the infrared light is able to penetrate deep into the skin and tissue and promote blood flow and therefore provide improved oxygenation as well as a greater influx of antibodies to the site. This phenomenon, in conjunction with the increased collagen production, results in improved skin repair and cellular regeneration, boosts new cell growth, enhances skin rejuvenation and stimulates various cellular processes and increases rejuvenation.

The blue infrared light has similar benefits to red infrared light with regards to increasing blood flow and its associated benefits. In addition, blue LED light therapy stimulates fibroblasts, the cells responsible for collagen production in the skin. This can help reduce the appearance of fine lines, wrinkles, and even scars. Advantageously, blue light has also been shown to have the most effective antimicrobial spectral range. This assists in killing various bacteria and fungi, allowing the skin to heal without secondary ailments such as fungal infection, acne and infections such as Staphylococcus. Blue light has also been shown to suppress cells that contribute to inflammation in the skin, which can also treat chronic inflammatory skin conditions such as eczema.

Embodiments of the wearable radiation emitting therapy device may employ a set of LED lights 150 broken up into regions as described above, with each region of LED lights including has both infrared, red and blue LEDs, positioned in alternating rows to allow for full coverage of all desired spectrums. In this regard, various types of LED bulbs may be placed in an alternating pattern so that the various wavelengths of light are evenly distributed to the treatment region. This type of arrangement may assist greatly with reducing overall inflammation.

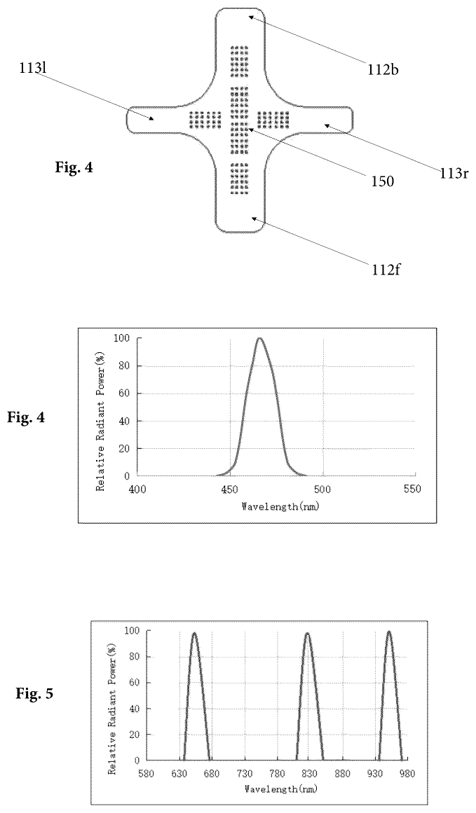

Referring now to FIG. 4, the wavelength emitted by the 470 nm LED light when operating at full relative radiant power is indicated. As is evident, the wavelength varies from around 450 nm to 490 nm as the light is operated. When operating at full capacity as it would be on the wearable radiation emitting therapy device, it emits light in the 470 nm wavelength spectrum.

Referring now to FIG. 5, the three-in-one LED light emits a spectrum around three target wavelengths. The exact wavelengths are achieved when the LED light is operating at full relative radiant power, as shown. These wavelengths are 660 nm, 830 nm, and 950 nm, respectively. It is contemplated, however, that these wavelengths may vary by roughly 20 nm in either direction as the LED lights reach their full power, where the exact frequency is achieved.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A wearable radiation emitting therapy device for an amputee patient, comprising:
a device body having an open top and an interior surface, wherein the device body is adapted to be positioned over an amputated member having an amputation site such that the interior surface is adjacent to at least the amputation site;
a plurality of electrical radiation emitting aspects positioned on the interior surface and configured to selectively generate radiation when activated, wherein the plurality of radiation emitting aspects are arranged such that when activated, the plurality of radiation emitting aspects direct radiation into the amputated member at the amputation site and into at least one area on the amputation member adjacent to the amputation site; and
at least one holding member integral with the device body, wherein the at least one holding member is configured to attach the device body to the amputated member.

2. The wearable radiation emitting therapy device for an amputee patient of claim 1, wherein the plurality of radiation emitting aspects are configured to selectively generate radiation at a plurality of wavelengths when activated.

3. The wearable radiation emitting therapy device for an amputee patient of claim 1, additionally comprising a control module operative to selectively activate the electrical radiation emitting aspects.

4. The wearable radiation emitting therapy device for an amputee patient of claim 1, wherein the plurality of radiation emitting aspects are defined by a set of LED lights.

5. The wearable radiation emitting therapy device for an amputee patient of claim 4, wherein the set of LED lights are arranged in a cross formation.

6. The wearable radiation emitting therapy device for an amputee patient of claim 4, wherein the set of LED lights are separated into six discrete regions.

7. The wearable radiation emitting therapy device for an amputee patient of claim 6, wherein the set of LED lights are arranged in a cross formation.

8. A wearable radiation emitting therapy device for an amputee patient, comprising:
a device body having an open top defining a proximal end, a closed bottom defining a distal end, and a plurality of side walls, wherein the device body includes an interior surface which extends across the enclosed bottom and at least a portion of the plurality of side walls;
wherein the device body is adapted to be positioned over an amputated member having an amputation site such that the interior surface is adjacent to at least the amputation site;
a plurality of electrical radiation emitting aspects positioned on the interior surface and configured to selectively generate radiation when activated, wherein the plurality of radiation emitting aspects are arranged such that when activated, the plurality of radiation emitting aspects direct radiation into the amputated member at the amputation site and into at least one area on the amputation member adjacent to the amputation site;
at least one adjustment strap integral with the device body, wherein the at least one adjustment strap is configured to tighten the device body on the amputated member.

9. The wearable radiation emitting therapy device for an amputee patient of claim 8, additionally comprising at least one securing strap integral with the device body, wherein the at least one securing strap is disposed between a first side wall among the plurality of side walls and a second side wall among the plurality of side walls at a location distinct from the location of the at least one adjustment strap.

10. The wearable radiation emitting therapy device for an amputee patient of claim 8, additionally comprising a first securing strap integral with the device body and a second securing strap integral with the device body, wherein the first securing strap is disposed between a first side wall among the plurality of side walls and a second side wall among the plurality of side walls in a first orientation and at a location distinct from the location of the at least one adjustment strap and the second securing strap is disposed between the first side wall and the second side wall in a second orientation that is a mirror image of the first orientation.

11. The wearable radiation emitting therapy device for an amputee patient of claim 8, wherein the plurality of radiation emitting aspects are defined by a set of LED lights.

12. The wearable radiation emitting therapy device for an amputee patient of claim 11, wherein the set of LED lights are arranged in a cross formation.

13. The wearable radiation emitting therapy device for an amputee patient of claim 11, wherein the set of LED lights are separated into a plurality of discrete regions.

14. The wearable radiation emitting therapy device for an amputee patient of claim 13, wherein:
the device body includes a first side wall, a second side wall, a third side wall and a fourth side wall, all amongst the plurality of side walls; and
at least one region of LED lights in the set of LED lights is disposed on each of the first side wall, the second side wall, the third side wall and the fourth side wall.

15. The wearable radiation emitting therapy device for an amputee patient of claim 8, wherein the plurality of radiation emitting aspects are configured to selectively generate radiation at a plurality of wavelengths when activated.

16. The wearable radiation emitting therapy device for an amputee patient of claim 8, additionally comprising a control module operative to selectively activate the electrical radiation emitting aspects.

17. A wearable radiation emitting therapy device for an amputee patient, comprising:
a device body adapted to be positioned over an amputated member having an amputation site and enclose at least the amputation site;
a plurality of electrical radiation emitting aspects integral with the device body and configured to selectively generate radiation when activated, wherein the plurality of radiation emitting aspects are arranged such that when activated, the plurality of radiation emitting aspects direct radiation into the amputated member at the amputation site and into at least one area on the amputation member adjacent to the amputation site; and
at least one holding member integral with the device body, wherein the at least one holding member is configured to attach the device body to the amputated member.

18. The wearable radiation emitting therapy device for an amputee patient of claim 17, wherein the plurality of radiation emitting aspects are configured to selectively generate radiation at a plurality of wavelengths when activated.

19. The wearable radiation emitting therapy device for an amputee patient of claim 17, wherein the plurality of radiation emitting aspects are defined by a set of LED lights.

20. The wearable radiation emitting therapy device for an amputee patient of claim 17, wherein the at least one holding member is defined by at least one of an elastic securing strap and a wraparound adjustment strap.
